(19) United States
(12) Patent Application Publication
(10) Pub. No.: US 2023/0186454 A1
(43) Pub. Date: Jun. 15, 2023

(54) MACHINE VISION SYSTEM FOR INSPECTING QUALITY OF VARIOUS NON-WOVEN MATERIALS

(71) Applicant: Valco Cincinnati, Inc., Cincinnati, OH (US)

(72) Inventors: David Williams, Cincinnati, OH (US); David Hughes, Cincinnati, OH (US); James Brashear, Cincinnati, OH (US); Harsh Shah, Cincinnati, OH (US); Nicolaie Stelian Vasiu, Cincinnati, OH (US)

(73) Assignee: Valco Cincinnati, Inc., Cincinnati, OH (US)

(21) Appl. No.: 18/065,131

(22) Filed: Dec. 13, 2022

Related U.S. Application Data
(60) Provisional application No. 63/288,761, filed on Dec. 13, 2021.

(51) Int. Cl. G06T 7/00; G06T 7/70; G06T 7/60

(52) U.S. Cl. CPC G06T 7/0004; G06T 7/70; G06T 7/60; G06T 2207/30204; G06T 2207/30108

(57) ABSTRACT

A method for inspecting a non-woven material is provided. The method comprises introducing, by a sonotrode, ultrasonic energy to weld the non-woven material, supporting, by an anvil roll, the non-woven material between the anvil roll and the sonotrode, rotating the anvil roll to transfer the non-woven material in a downstream direction, taking, by an imaging unit disposed downstream of the anvil roll, an image of the non-woven material, and processing the image by an image processing unit. The image processing unit comprises identifying a defect based on the image, classifying the defect into one of a plurality of categories, analyzing a cause of the defect based on the respective category, determining a component causing the defect, and notifying the cause with a recommended remedial action associated with the component.

### TECHNICAL FIELD

The present specification generally relates to camera-based devices for inspecting defects and other attributes on discrete products or webs of various materials for assessing the quality and presence of the bonds (e.g., weld bonds) and other attributes.

### BACKGROUND

Current equipment for producing, for example, non-woven materials (e.g., laminated non-woven materials) for use in such disposable articles as face masks, absorbent pads, diapers, and feminine-care products operates largely open loop with no inspection of the products produced. Products are sometimes produced in web form and sometimes as discrete products. In one form, laminations are formed using ultrasonic bonding (also referred to as ultrasonic welding). Web forms are often rewound back onto a large roll where it is difficult to inspect for defects inside this roll. Devices such as cameras are sometimes found on such machines, but they are usually simple systems that provide the user a constantly-updating picture of what is going on, but no substantial evaluation of its quality. Sometimes the webs contain elastic material in between multiple layers of non-woven or other material. The elastic material may need to be verified to be present and in the proper position. The thickness, density or consistency and quality of the non-woven material itself also may need to be inspected.

### SUMMARY

According to the subject matter of the present disclosure, ultrasonic bonding may be used as a method of attaching multiple layers of materials. Inspecting products (e.g., rolls or discrete products) produced using the ultrasonic bonding method may require determining bond locations and defect types (e.g., repeating defects, continuing defects, or random defects). In addition, detecting whether the thickness or “basis weight” of the rolls of discrete products after bonding is truly as it should be or is too thin or thick. Also, detecting the dimensions of the products may help determining defects. Once certain defects are determined, determining potential root causes of the defects for inspection is also beneficial.

In the field of bond inspection, some equipment is capable of basic bond location measurement of the articles, but not able to determine what types of defects are present. This lack of effective inspection equipment may contribute to large amounts of waste being produced if a problem occurs, as well as machine downtime related to stopping the downstream article manufacturing process to remove defective welded materials.

In accordance with one embodiments of the present disclosure, a method for inspecting a non-woven material is provided. The method comprises introducing, by a sonotrode, ultrasonic energy to weld the non-woven material, supporting, by an anvil roll, the non-woven material between the anvil roll and the sonotrode, rotating the anvil roll to transfer the non-woven material in a downstream direction, taking, by an imaging unit disposed downstream of the anvil roll, an image of the non-woven material, and processing the image by an image processing unit. The image processing unit comprises identifying a defect based on the image, classifying the defect into one of a plurality of categories, analyzing a cause of the defect based on the respective category, determining a component causing the defect, and notifying the cause with a recommended remedial action associated with the component.

In accordance with another embodiment of the present disclosure, a method for inspecting defects on a non-woven material is provided. The method comprises introducing ultrasonic energy to weld the non-woven material, transferring the non-woven material in a downstream direction, taking an image of the non-woven material after welding the non-woven material, identifying a defect based on the image, classifying the defect into one of a plurality of categories, analyzing a cause of the defect based on the respective category, determining a component causing the defect, and notifying the cause with a recommended remedial action associated with the component.

In accordance with yet another embodiment of the present disclosure, a method for inspecting a non-woven material is provided. The method comprises introducing, by a sonotrode, ultrasonic energy to weld the non-woven material, supporting, by an anvil roll, the non-woven material between the anvil roll and the sonotrode, rotating the anvil roll to transfer the non-woven material in a downstream direction, taking, by an imaging unit disposed downstream of the anvil roll, an image of the non-woven material, and processing the image by an image processing unit comprising identifying a defect based on the image, classifying the defect into one of a plurality of categories, analyzing a cause of the defect based on the respective category, determining a component causing the defect, and taking a recommended remedial action associated with the component.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 is an illustrative drawing of a system for inspecting a non-woven material, according to one or more embodiments described herein;

FIG. 2 depicts details associated with the system including an anvil roll, a sonotrode, and an imaging device of the system of FIG. 1, according to one or more embodiments described herein;

FIG. 3 depicts certain types of defects that may be identified by the system of FIG. 1, according to one or more embodiments described herein;

FIG. 4 depicts close-up photographs of sample defects that may be identified by the system of FIG. 1, according to one or more embodiments described herein;

FIG. 5 depicts repeating defects generated from a damaged anvil pin of an anvil roll of the system of FIG. 1, according to one or more embodiments described herein; and

FIG. 6 depicts a variety of types of defects that may be identified in the system of FIG. 1, according to one or more embodiments described herein.

### DETAILED DESCRIPTION

Various embodiments described herein may identify defects and provide recommended remedial actions associated with the defects of ultrasonic welding or related bonding systems. The system may include a computer, a camera, an encoder, lighting, a marking valve, as well as signal processing to identify and track weld defects and to classify the defects formed on a continuous web of non-woven material. The system may provide a machine vision system with machine learning functionality such that the defects may be classified to allow for a determination of the cause of the defect, as well as provide assistance in maintenance of the system by providing a recommended remedial action. The system may also log the length of the non-woven material to enable the ability to map the finished roll for further quality controls to be implemented. Moreover, the particular configuration may be modular, including multiple reconfigurable equipment types that are used to take advantage of certain aspects of the material being inspected. Various features and details are explained more fully with reference to the non-limiting embodiments that are illustrated in the accompanying drawings and detailed in the following description.

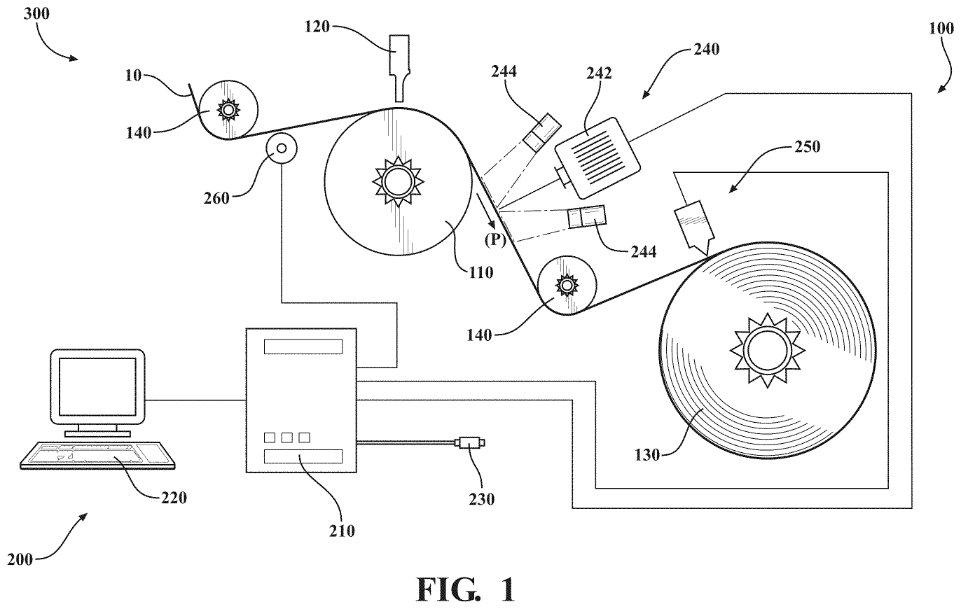

Referring initially to FIG. 1, a system 300 for inspecting a non-woven material 10 according to one embodiment of the present disclosure includes a welding system 100 and an image inspection system 200. In embodiments, the system 300 may be used to produce discrete articles or the like. While the system 300 is depicted as a web-based imaging system for manufacturing elastic portions of an article such as a diaper, other operations, such as cutting and welding onto the finished diaper, may take place on a different machine that is not presently shown. As such, the system 300 may convert the non-woven material 10 into discrete articles with or without additional material layers.

The welding system 100 includes a sonotrode 120 and an anvil roll 110. The sonotrode 120 introduces ultrasonic energy to weld the non-woven material 10. The sonotrode 120 may heat the anvil roll 110 such that a portion of the non-woven material 10 is briefly heated and melted by the ultrasonic energy in order to form bonds or welds. The anvil roll 110 may have a plurality of pins that may be heated by the ultrasonic energy. The pins may be disposed on the anvil roll 110 on a surface facing the non-woven material 10. The pins may be disposed in a pattern corresponds to a welding pattern to be formed on the non-woven material 10. The anvil roll 110 is disposed on the opposite side of the sonotrode 120 such that it supports the non-woven material between the anvil roll 110 and the sonotrode 120. The anvil roll 110 rotates to transfer the non-woven material 10 in a downstream direction (e.g., a travel path (P)). The welding system 100 may further include one or more transferring rolls 140 supporting and/or transferring the non-woven material 10, and a roll 130 winding the non-woven material 10 after welding. The welding system 100 may work in conjunction with the image inspection system 200 such that detection of defects identified by the image inspection system 200 may be used to adapt operation of the welding system 100, which will be discussed in detail later.

The image inspection system 200 includes an imaging unit 240, an image processing unit 210, and a marking valve 250. The imaging unit 240 is operable to take an image (e.g., a photograph, a video, or the like) of the non-woven material 10. The imaging unit 240 may include a camera 242 and lighting 244. The camera 242 may take images of the surface of the non-woven material 10. The camera 242 may be set to focus on the surface of the non-woven material 10. The camera 242 may be either a contact image sensor line-scan camera, a traditional line camera, or any type of camera that may take images of the non-woven material 10. The lighting 244 may be either built into the camera 242 or be configured as an external lighting source separate from the camera 242. The camera 242 may be set to take images covering the entire width of the non-woven material 10 or a certain area on the non-woven material 10 that is less than the entire width of the non-woven material 10.

The imaging unit 240 is disposed downstream of the sonotrode 120. Therefore, the imaging unit 240 may acquire images of the non-woven material 10 after passing a region between the anvil roll 110 and the sonotrode 120 where the non-woven material 10 is welded. The transferring rolls 140 and the anvil roll 110 may be arranged to support the non-woven material 10 to straighten the non-woven material 10. The imaging unit 240 may be arranged to take images of the non-woven material 10 in a location where the non-woven material 10 is straightened (i.e. is generally planar). The camera 242 may provide images better suitable for inspection when the non-woven material 10 is straightened.

The imaging unit 240 may be connected to the image processing unit 210 through wired or wireless communication links. Connection between the imaging unit 240 and the image processing unit 210 may include the CoaXPress interfacing standard, which in turn may utilize a CoaXPress Framegrabber/Digitizer in order to transfer an acquired image to memory of the image processing unit 210. The connection between the imaging unit 240 and the image processing unit 210 may include the CameraLink connection, which is a common method of communicating camera data. Connection between the imaging unit 240 and the image processing unit 210 may include the gigabit ethernet interfacing standard, which in turn may utilize a gigabit ethernet connection in order to transfer the acquired image to the memory of the image processing unit 210.

The image processing unit 210 may include one or more processors, computer-readable media in the form of memory and input/output (I/O), all signally coupled via bus. The processor may include modules to perform various arithmetic, control logic and related processing functions. In one form, the memory—which may be connected to the bus by one or more data media interfaces—in readable or writable form may include both volatile media (such as random access memory, RAM, cache or the like, in one form for the storage of data) and non-volatile media (such as read-only memory, ROM, often in the form of flash, hard disks, optical disks such as compact disks (CDs), digital video disks (DVDs) or the like, in one form for the storage of programs for various algorithmic control logic-based operations), as well as in removable and non-removable media configurations. In one form, the input portion of the I/O is configured as a keyboard, mouse, verbal command, joystick or other known means, while the output portion may be in the form of video display, audio message or other known modality. Although the bus may be configured as a wired connection, it will be appreciated that it may be configured to have at least some of its functionality embodied in a wireless format (including the transmitting or receiving of radio-frequency signals via suitable radio-based chipset and antenna), and that all such variants are deemed to be within the scope of the present disclosure.

One or more programs to carry out one or more operations or methodologies as discussed herein may be stored in the memory. By way of example, and not limitation, such programs may include an operating system, one or more application programs, program data or the like, as well as programs for network connectivity such as through a suitable communication protocol along with a suitable network adapter or other bus-enabled interface. In one form, such connectivity may be through a distributed architecture. In such a configuration, remote environments such as a cloud (not shown) may be used for one or more of control, operation, data storage, analytics or the like. In addition, such a distributed configuration may include the use of various modules making up one or more parts of the program or programs; these may be located in both local and remote computer system storage media, including those discussed herein. Regardless of whether the program or programs are formed as part of a distributed or singular architecture, the image processing unit 210 may provide various functionalities.

The image processing unit 210 may further track identified defects, mark the non-woven material 10, stop one or more of components of the welding system 100, and create one or more of an alarm, notification, or message in the event that determination of a defect is identified. In one form, the program or programs may be in modular format such that each module generally carries out one or more discrete functions or tasks of the functions and/or methodologies of embodiments of the welding system 100 and the image inspection system 200 as described herein. These modules may include—among other things—various instruction sets, logic, programs, routines, objects, components, data structures or the like in order to perform particular tasks or implement particular abstract data types. Furthermore, such modules may be configured as a hardware module, a software module or a combination of both.

In embodiments, various machine learning, training, testing, validation, and deployment may be utilized by the image processing unit 210. The image processing unit 210 may be configured as a central processing unit (CPU) for conventional processing tasks, as well as high-power graphics cards and processors (such as field-programmable gate arrays (FPGAs), application-specific integrated circuits (ASICs), graphics processing units (GPUs) or the like) in situations where large amounts of matrix multiplication or related data processing (such as when used by a neural network) may be required. Machine learning functionality (and its learn-by-example or learn-by-observation modes of operation) may be understood to be in addition to, or a replacement for, the mode of operation embodied in the image processing unit 210 through its components and program or programs.

The image processing unit 210 may be coupled to a user interface 220 and a network connector 230 (e.g., a wireless network connector or a wired network connector), or the image processing unit 210 may include the user interface 220 and/or the network connector 230. The user interface 220 may receive user input from a user, display images taken from the imaging unit 240, and/or display an image or a video or generate a sound associated with a notification of the cause with the recommended remedial action. The user interface 220 may provide the notification with explanations or instructions which a user may follow to remedy the identified defect.

It is noted that the image inspection system 200 may be configured as a centralized or distributed architecture depends on numerous factors. For example, the use of a centralized system may be beneficial for certain tracking operations, while a distributed system could be more practical for certain forms of data acquisition, communication and control.

The image inspection system 200 may employ machine learning models and neural networks (particularly those with deep learning functionality) in order to identify a defect based on the acquired images, classify the defect into one of a plurality of categories, analyze a cause of the defect based on the respective category, and notify a user of the cause with a recommended action associated with the cause or employ the recommended action. Particular examples of neural network approaches that may be used to assist the image inspection system 200 with recognition of the acquired images may be achieved with convolutional neural networks (CNNs), recurrent neural networks (RNNs), long short-term memory neural networks (LSTMs) or the like.

A CNN-based machine learning model—with its ability to mimic the hierarchical structure of a human-based vision system through feature extraction and related classification-based image recognition—is particularly useful for machine vision-based bond defect identification. For example, a CNN-based approach may be trained with baseline bond defect images and one or more machine learning algorithms such that the resulting machine learning model can produce labeled outputs to recognize images of various bond defects, as well as to categorize such defects by severity or nature. In another example, an RNN-based or LSTM-based approach may be trained to provide relationship data across images through a timeframe, in order to categorize such bond defects as recurring as opposed to random. In another example, machine learning libraries [such as through TensorFlow (perhaps in conjunction with a classification builder such as TFLearn), PyTorch, Scikit-learn or the like] of images may be used in order to help classify the defect.

It will be appreciated that in order to improve the image classification functionality of a machine vision system that employs a neural network-based machine learning model (such as through reduced processing time) into the image inspection system 200, loss function minimizations may be achieved through gradient descent-based methods (including stochastic gradient descent—typically in conjunction with a backpropagation algorithm—for larger acquired image data sets); such methods may be implemented in known libraries such as those previously mentioned. Relatedly, processing time and memory use requirements within the image processing unit 210 may be significantly improved if the machine vision system employs a dimensionality reduction technique to the acquired data, particularly if some of the features (that is to say, input variables) can be prioritized by their relative importance. In one form, linear discriminant analysis (LDA) may be used, while in another, principal component analysis (PCA) may be used, while in still another, a kernel-based approach, such as kernel discriminant analysis (KDA) may be used.

In embodiments, a neural network-based machine learning model, such as the CNN, may be included as part of the image inspection system 200 which may act as such a machine vision system. In the machine vision system, various modules, including those for image acquisition, preprocessing, segmentation, feature extraction, classification and associated contextual processing, may be used in cooperation in order to train, test, validate and deploy the machine learning model. For example, the images are processed according to a series of vision algorithms to establish what bonds are good and which ones are defects. These algorithms may include (in addition to the aforementioned classification) localization capability or multiple object detection capability. With the former, not only is the nature of the bond defect classified, but its position on the web as well can be ascertained, while with the latter both the nature and location can be ascertained. Once the images have a count of defective bonds, a determination can be made of the type and quantity of defects on the revolution and whether this portion of the web or discrete product should be marked or noted in a roll map (e.g., an image of the surface of the non-woven material 10 corresponding to the outer surface 112 of the anvil roll 110). In one form, the machine vision system may include controller-based feedback features in order to regulate some or all of the production of non-woven materials for disposable articles, including control over the welding system 100.

Although neural networks, in general, and CNNs, RNNs and LSTMs, in particular, are discussed in the present disclosure, it is understood that other classification-based models may be employed, including those invoking decision trees or ensemble-based approaches, such as random forests or the like, as well as unsupervised clustering-based approaches such as k-means or the like. These models and their ways of handling the acquired data through corresponding algorithms and associated tools as a way to produce a finished prediction, use or inference are deemed to be within the scope of the present disclosure.

Other components such as programmable logic controllers (PLCs) may be used in order to automate the production and detection process. Furthermore, particularly in situations where low data-rate sensing of certain operational parameters may be used, the PLCs may be software-based such that the welding system 100 and the image inspection system 200 operate as part of a supervisory network of machine controls. This in turn permits simpler integration into existing machine controls and allows actions such as bonding parameter adjustments, web guiding adjustments or the like to be made live based on feedback from the machine vision system. Moreover, such a system may be coupled with analytics derived from sensed and other data in order to evaluate historical and baseline trends, as well as to provide predictive capability in order to alert a user that adverse changes to the system may be imminent such that corrective maintenance or related measures may be taken. In one form, the predictive analytics may use a machine learning model in conjunction with various forms of sensor-acquired data. Moreover, real-time collection and analysis of the data made possible with the image inspection system 200 may be made available to production control decision-making, whether automated or with a human in the loop. In such case, conveyance of such data or analysis may be made through the network connector 230, such as wired networks (e.g., local wired networks, the ethernet or the like) or wireless networks (e.g., WiFi, cellular or other radio-based mechanism using high-bandwidth or low-bandwidth transmission protocols). Moreover, a virtual private network (VPN) may be set up in order to securely convey such data, analytics, control instructions or corrective action.

The machine vision system may be configured as a distributed platform using one or more of the aforementioned machine learning models and their related algorithms. In such case, outfitting the image inspection system 200 (possibly along with the welding system 100) with the aforementioned intelligence that could be built into the imaging unit 240 or related end nodes in order to provide so-called Internet of things (IoT) functionality and could help promote decentralized operation and associated autonomy within a production environment, including the capability to perform edge-based data acquisition, processing and associated analytics.

The marking valve 250 is disposed downstream of the imaging unit 240. The marking valve 250 is configured to provide marking, flagging, or related indicia on the non-woven material 10. The marking valve 250 may make a mark, either physically or digitally, on the non-woven material 10 based on a signal from the image processing unit 210. For example, the marking valve 250 may be a web inserter, which inserts a small piece of paper into the roll (e.g., between the layers of the non-woven material 10 of the roll 130) or create a flag in stored image data of the non-woven material 10. The flag may be displayed on the roll map.

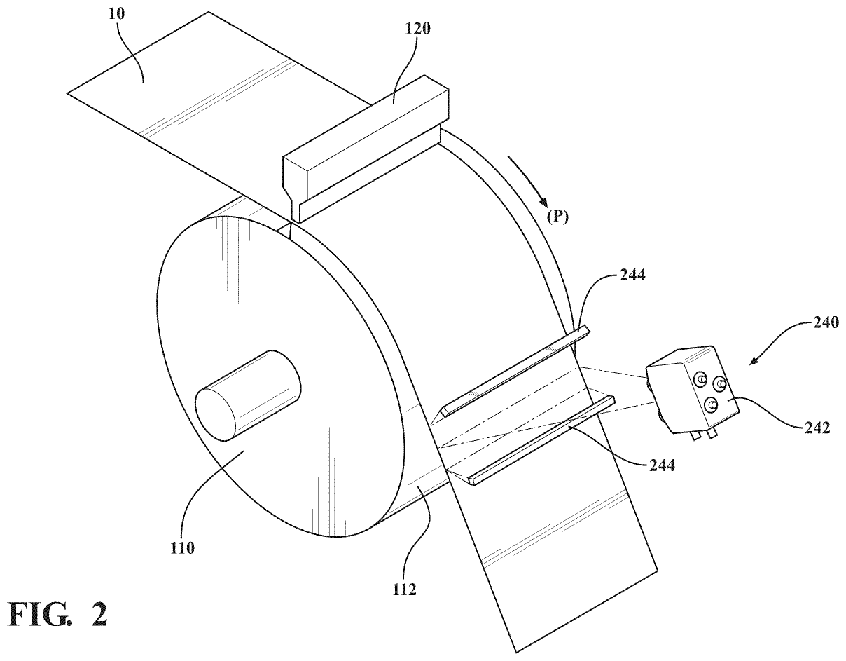

Referring to FIG. 2, details of the anvil roll 110, the sonotrode 120, and the imaging unit 240 are shown according to embodiments. In embodiments, the camera 242 of the imaging unit 240 is placed downstream of the sonotrode 120. The anvil roll 110 rotates to transfer the non-woven material 10 in the downstream direction (e.g, the direction of the travel path (P)) and has raised pins (not shown) on an outer surface 112 of the anvil roll 110 supporting the non-woven material 10 to form a bond pattern on the non-woven material 10. Sonic energy travels from the sonotrode 120 to the pins such that a localized portion of the non-woven material 10 is briefly heated and melted in order to form the bond or weld. The lighting 244 in embodiments may be a separately mounted light module that illuminates the surface of the non-woven material 10 from the top, thereby allowing the camera 242 to take images for detecting the associated features and assess bond locations, material quality as well as measure any critical dimensions as needed. In embodiments, the imaging and analysis of the non-woven material 10 takes place continuously while the non-woven material 10 as it is moving along the travel path (P).

In embodiments, the camera 242 is a line-scan camera, such as a Basler Racer type, that takes a view of the non-woven material 10 through an attached lens. The camera 242 may image one pixel row across the non-woven material 10 at a time. Line-scan images may be stitched together such that every bond point may be entirely inspected. The images of each line-scan over a revolution of the anvil roll 110 may be combined to be analyzed as a unit and to keep track of the location of defects along the non-woven material 10. The camera 242 may capture a broad width and focus of the images down to a very small width of the pixel array. More than one camera 242 may be placed into visual communication with various parts of the system 300 in order to inspect numerous locations along the process. The combination of images of numerous locations from cameras 242 may processed to be combined, which may enhance accuracy. In embodiments, the camera 242 may include a zooming feature such that a view of the relevant portions of the non-woven material 10 can be enhanced as needed, thereby allowing a user (in manual modes of operation) or the image processing unit 210 (in automated modes of operation) to ascertain more information associated with the images. The camera 242 may be a Mitsubishi contact image sensor type KD-6R.

The lighting 244 may be disposed at an acute angle (for example, at opposing 45° angles of incidence) or any angle suitable to illuminate the face of the non-woven material 10 while casting very few shadows of the fibrous material contained therein. In another form, the lighting 244 is arranged in a manner to illuminate the non-woven material 10 from behind to cause backlighting (such as dark field back lighting). Backlighting may illuminate the non-woven material 10 without providing direct illumination of the source into the camera 242. The lighting 244 may be LED or fluorescent in order to create even illumination across the entire non-woven material 10. Appropriate lensing may be added to spread the light out and ensure that there are no bright and dim spots; as such, wavelength, angle and placement may be varied in order to optimize defect identification. The lighting 244 may have width from 100 mm to 1000 mm wide depending on the application.

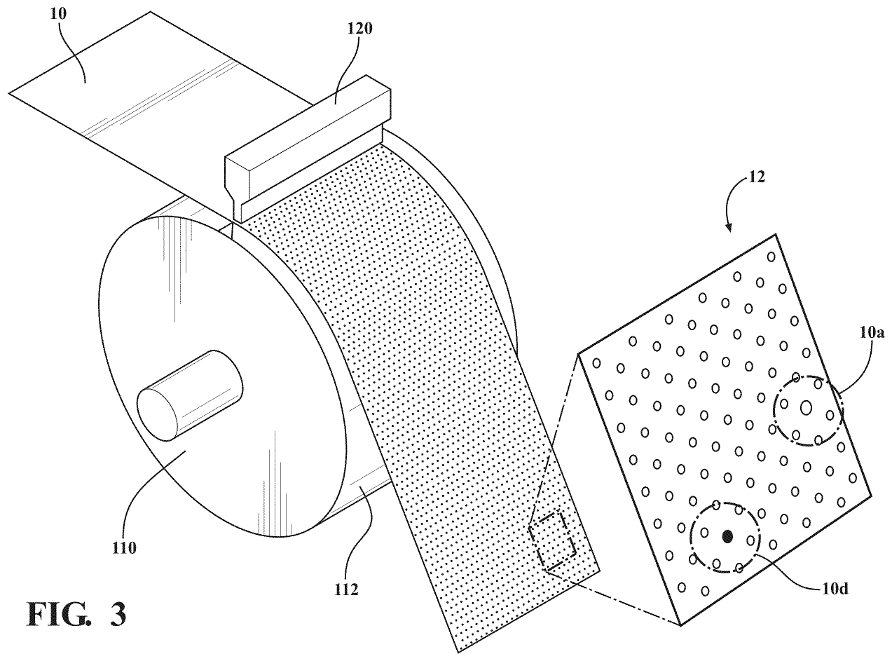

Referring to FIG. 3, the image processing unit 210 is configured to identify a defect. Certain types of bond defects (e.g., bonds 10a and 10d) are shown that may be identified as defective by the image processing unit 210. The defect may be identified based on one or more images taken by the imaging unit 240. The image processing unit 210 receives the images from the imaging unit 240 and processes them to identify the defect. The image processing unit 210 analyzes the images to further determine the location of the defective bonds 10a and 10d formed in the non-woven material 10. The image processing unit 210 may compare the images from the imaging unit 240 to one or more baseline test images to identify the defect and/or may analyze the individual bonds to determine whether the individual bonds meet certain requirements to be non-defective. In all cases, acquired images may be stored in memory, as can databases of numeric values representing the type of defect, the quantity of such defects and the frequency of defects, as well as establishing a particular spatial coordinate location on the non-woven material 10 where such defects are identified. In embodiments, only the images including the bonds identified as defective may be stored in memory.

The images may be provided at multiple different resolutions, such as, but not limited to, 600, 300, 150 or 75 dpi. The images may be acquired with a high resolution and processed at lower resolutions initially to help promote real-time operation, while later preserving the higher resolution archived images for better diagnostics. For example, if an image is acquired at 600 dpi, such equipment has the ability to selectively remove the pixels and save portions at higher resolutions. This allows the basic image analysis to take place on a lower resolution image but still have available all or portions of images for higher resolution for human viewing and “zooming” functions. Standard line-scan cameras can also have higher resolutions and do the same technique of having, for example, a 4096 pixel imager and only keep every fourth pixel for analysis.

The images may be displayed on a display (e.g., the user interface 220) such that a user (e.g., a system operator or the like) may manually inspect the images. In embodiments, only the images including the bonds identified as defective may be displayed. In embodiments, the image processing unit 210 may be configured as a server to display the data on a web browser. In embodiments, the images may be accessed by connecting remotely (e.g., through the network connector 230) to the image processing unit 210 and viewing the defects remotely on a display. The display may display a graphical representation of a roll map (e.g., an image of the surface of the non-woven material 10 corresponding to the outer surface 112 of the anvil roll 110). The roll map may show the location of the defects identified by the image processing unit 210.

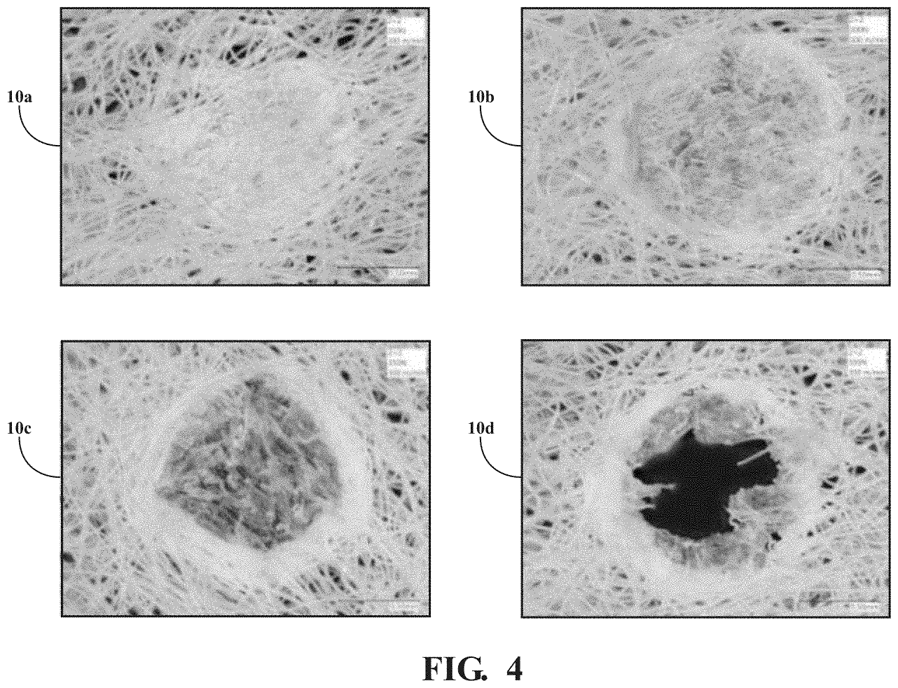

Referring to FIG. 4, exemplary images of close-up photographs of bonds are depicted. For example, each of bonds 10a to 10d represents a different quality level (e.g., a degree of defect). The non-woven material 10 may be bonded to a layer of material (e.g., one or more laminate layers). In embodiments, the image processing unit 210 may use algorithms, whether of the rule-based or machine learning variety, that are associated with the image processing unit 210 to evaluate the strength and other indicia of the bond quality. For example, a missing bond may show no change at all as no bond is formed in the non-woven material 10. The exemplary images depicted in FIG. 4 are provided as examples, and they may appear different from the images actually taken by the imaging unit 240.

In embodiments, the image processing unit 210 may determine a degree of the defect of the bonds. The image processing unit 210 may determine the degree of the defect based on certain criteria associated with the bond. For example, the criteria may include the size and/or the shade of the bonds. The degree of defect may be determined based on whether the criteria may meet threshold values. The degree of defect may be determined based on comparison between the criteria and standard values. The threshold values and/or the standard values may be changed based on the material type, the weight or thickness (e.g., a basis weight) of the non-woven material 10, and/or bond size and geometry. For another example, the images of the bonds may be compared with the standard images to determine the degree of the defect of the bonds. The images may be compared to determine dissimilarities to identify defects and/or determine the degree of defect.

A weak bond 10a and a burn-through bond 10d may be identified as defective by the image processing unit 210. The weak bond may be lighter in shade and/or smaller in size than the average measures of shade and/or size of the bonds when the non-woven material 10 is front lighted as can be seen in the photograph of the weak bond 10a. In embodiments where the non-woven material 10 is backlighted, the weak bond may look darker in shading and/or smaller in size than the average bond. The weak bond may indicate either insufficient heating time or heating concentration on the non-woven material 10. The weak bond may also indicate that the non-woven material 10 has not properly contacted the pins on the anvil roll 110 in order to allow the non-woven material 10 to sufficiently melt. The burn-through bond 10d may be formed when the heat applied to the bond is high enough to fully melt the non-woven material 10 such that a hole is formed. While the melted areas are still bonded together, the strength of the burn-through bond 10d is lower than a good bond 10b or a fair bond 10c, which does not form a hole but forms a melted area in a middle portion thereof. The burn-through bond 10d may be identified by highlighting visual features associated with the size and shade of the bond. In embodiments where the non-woven material 10 is backlighted, the burn-through bond 10d may be shown to be darker in shade, and the size of the entire bond may be larger than the average measured size of the bonds.

The good bond 10b and the fair bond 10c may be identified as non-defective bonds by the image processing unit 210. The good bond 10b may have a rim around the bond with a slightly shaded inside which may indicate a balance of heat application without damaging the laminate layers and provide enough melting of the non-woven material 10. The fair bond 10c may have a rim more visible than that of the good bond, which may be evidence that the non-woven material 10 starts to build up around the edge of the bond rather than staying in the middle of the bond. As a result, the strength of the adhesion of the fair bond between the non-woven material 10 and other layers may be less than the good bond. Also, the risk that the non-woven material 10 and the other layers will come apart is higher than the good bond. However, the fair bond may provide enough bond strength to the non-woven material 10 compared to the defective bonds (e.g., the weak bond 10a and the burn-through bond 10d).

The identification of the defective bond may be set up differently than the above exemplary embodiments depending on the purpose of welding and/or bonding of the non-woven material 10. For example, only the weak bond 10a may be identified as defective while the good bond, 10b, the fair bond 10c, and the burn-through bond 10d may be identified as non-defective. For another example, the weak bond 10a, the good bond 10b, and the fair bond 10c may be identified as non-defective and only the burn-through bond 10d may be identified as defective.

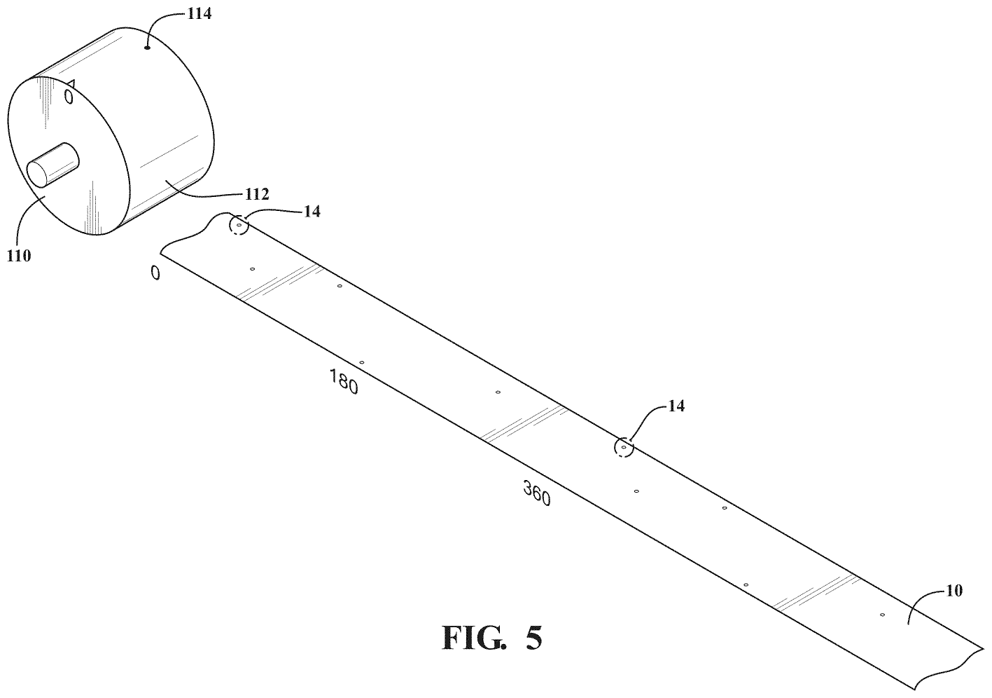

Referring to FIG. 5, a portion of the non-woven material 10 is shown unrolled such that repeating locations of defects 14 can be seen on the non-woven material 10. The defects 14 are originated from a damaged or contaminated portion 114 on the outer surface 112 of the anvil roll 110. For example, the repeating locations of the defects 14 on the non-woven material 10 is formed by the portion 114. The portion 114 may repeatedly form the defects 14 on the non-woven material 10. Each location of the defects 14 may correspond to the location of the portion 114. Defect data associated with the defects 14 may be received by or stored to the image processing unit 210, and the defect data of the defects 14 may be compared to a threshold (e.g., a threshold frequency, a threshold distance between the locations, a threshold number of occurrences, or the like). The threshold may be set by the user or calculated from threshold data stored in the image processing unit 210. The welding system 100 may operate based on the comparison between the defect data and the threshold data. For example, when the number of occurrences of the defects 14 exceeds the threshold, the welding system 100 may be signaled or stopped. The image processing unit 210 may signal the marking valve 250 to leave a mark on the non-woven material 10 at a location corresponding to the defects 14 based on the comparison between the defect data and the threshold data. For example, the marking valve 250 may mark the location of all or some of the defects 14.

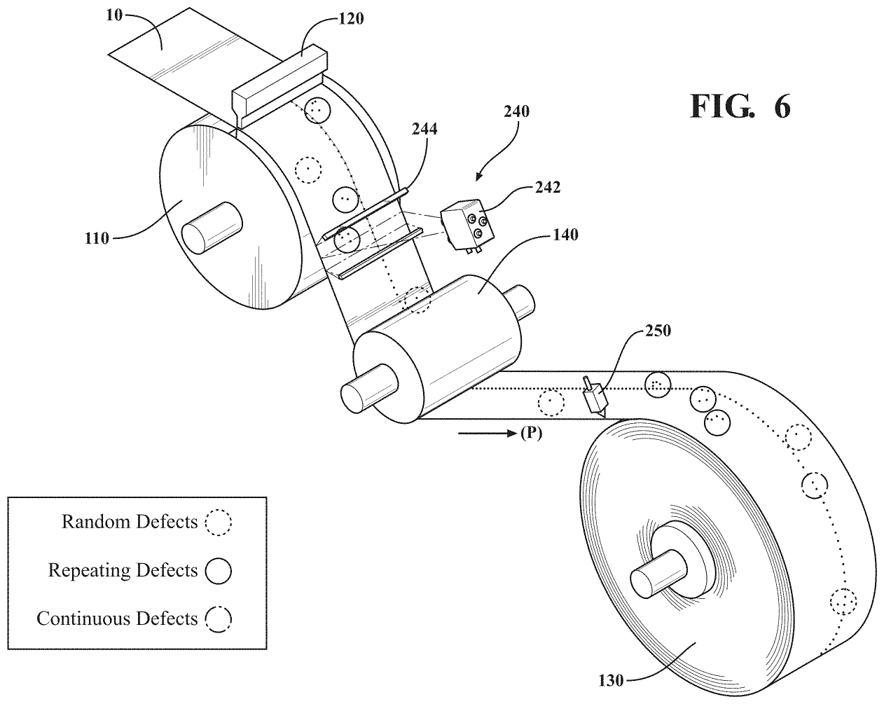

Defects are not limited to repeating defects and may include different types of defects. As non-limiting examples, referring to FIG. 6, various defects appear on the non-woven material 10. The image processing unit 210 is configured to classify the defects into one of a plurality of categories. The categories may include random defects, repeating defects, and continuous defects. The random defects are circled in a dotted line, the repeating defects are circled in a solid line, and the continuous defects are circled in a dash-dotted line. The random defects may be randomly formed on the non-woven material 10. The repeating defects (e.g., the defects 14 in FIG. 5) may occur repeatedly formed on the non-woven material 10. The continuous defects may occur continuously on the non-woven material 10 (e.g., continuous dots along the direction of the travel path (P) at a middle portion of the non-woven material 10). Certain types of repeating defects may be classified as the random defects when the repeating defect slips its location and the defects are not corresponding to a specific location of the anvil roll 110. For example, certain repeating defects with a slip tolerance larger than a standard tolerance may be classified as random defects. The continuous defects may be generated all along the non-woven material 10, which may be easily detected by their continuous nature. Additionally, defects associated with alignment of the non-woven material 10 may be classified as alignment defects. Additionally, the categories may include non-repeating defects as well as columnar defects, which may be also be classified by the defect type (e.g., not present, weak, burn-through, etc.). The columnar defects may be caused by a problem with the sonotrode 120.

The classified defects may be further analyzed to determine a cause of the defects. The image processing unit 210 is configured to analyze a cause of a defect based on the respective category of the defect. The image processing unit 210 may be further configured to notify the cause with a recommended remedial action associated with the cause. The recommended remedial action is beneficial in that it may help avoid the waste associated with producing multiple rolls or multiple quantities of products when defects are detected. The image processing unit 210 may cooperate with the welding system 100 to perform the recommended remedial actions such as interruption or adjustment of the welding operation. For example, the image processing unit 210 may send a signal to the welding system 100 to take the recommended remedial action associated with the cause. The image processing unit 210 may be configured to stop or continue operation of the welding system 100 based on the recommended remedial action associated with the cause. The recommended remedial actions may further include—among other things—tracking the defect to a known location on the non-woven material 10, moving the non-woven material 10 laterally, which may include moving the transferring rolls 140 and/or the anvil roll 110, as well as stopping the operation of the welding system 100 altogether. The notification may be provided via the user interface 220. The remedial measure may include altering the parameters of the sonotrode 120 that may affect the strength of the bond.

The image processing unit 210 may use heuristics and statistical methods to analyze the cause of the defects. For example, the random defects may result from the nature of the ultrasonic welding process, as well as from random variations in the material (e.g., the non-woven material 10, the additional layers, or the like). The random defects may occur as a result of basis weight issues, which may show up as deviations in the brightness of the image and the contrast of the brightness to neighboring areas of the non-woven material 10. Where there are large changes in the image density, the random defects with the basis weight of the non-woven material 10 may be detected. Therefore, the random defects may result from basis weight issues of the non-woven material 10. The random defects may be indicative of occasional weak or burned-through bonds which tend to occur randomly due to material variations and natural variations in the ultrasonic welding process. Therefore, the presence of a number of random defects may be treated with less severity compared to the repeating defects or continuous defects.

The random defects may be notified to the user with a recommended remedial action associated with the cause of the random defects. The recommended remedial action may be to continue operations because the causes of the random defects may be possibly ignored for a period of time. The recommended remedial action may be to change the basis weight of the non-woven material 10 when the cause of the random defect is determined to be the basis weight of the non-woven material 10. When the cause is width variation of the non-woven material 10, the recommended remedial action may be inspecting the non-woven material 10.

The repeating defects may be caused by damage or contaminations on the anvil roll 110, the sonotrode 120, or other components of the welding system 100, which may form defects repeatedly at a particular location. The repeating defects that occur in significant quantity may warrant taking an appropriate action. For example, the repeating defects occurring more than a threshold frequency may require maintenance to the anvil roll 110 and/or the sonotrode 120. The remedial action for repeating defects may require only a particular point on the anvil roll 110 or other rolls to be maintained and repaired. The particular point may correspond to a pin or a raised potion on the anvil roll 110 or other components along the welding system 100. Because the same particular point causes the repeating defects, the location of the repeating defects monitored and/or stored may be analyzed to identify the location of the particular point causing the repeating defects. The recommended remedial action may be determined based on the identified location of the repeating defects or the identified location of the particular point. The notification may be provided when the severity of the repeating defects determined by the image processing unit 210 when the severity reaches a threshold value. The welding system 100 may be stopped based on the recommended remedial action and/or the severity of the repeating defects, which may require inspection of the components of the welding system 100 (e.g., the anvil roll 110).

For example, the image processing unit 210 may identify the location of the particular point causing the repeating defects based on the location of the repeating defects on the non-woven material 10. The location of the repeating defects on the non-woven material 10 may be determined based on location data from the encoder 260 and/or the imaging unit 240, which may provide location information of the rotational position of the anvil roll 110. Other positional measurement methods such as magnetic encoders, rotary variable differential transformers (RVDTs) or Hall effect devices may be used to identify the location of the particular point. The recommended remedial action may indicate the location of the particular point causing the repeating defects that need to be repaired.

The continuous defects may be caused by a defect in the sonotrode 120. The sonotrode 120 may be defective when an area of the sonotrode 120 is not properly emitting ultrasonic waves. The defective area of the sonotrode 120 tends to produce weak or no bonding. The defects caused by the sonotrode 120 may continuously appear on the non-woven material 10. For example, the continuous defects may be characterized by a continuous row of defective bonds. The recommended remedial action for the continuous defects may be associated with the sonotrode 120. For example, the recommended remedial action may be repairing, replacing, or cleaning the sonotrode 120.

The alignment defects may be caused by varying width of the non-woven material 10. Additional layers bonded with the non-woven material 10 may also have varying width. The width variation may be measured and compared with an allowable tolerance (e.g., a threshold width). When the width variation of the non-woven material 10 or the additional layers are determined to be greater than the allowable tolerance, the location with the varying width may be marked by the marking valve 250. Depending on the width variation, the recommended remedial action may be stopping the welding system 100 for any necessary maintenance.

As with the various categories of defects as described above, the intelligence gleaned from the machine vision system, whether through a priori or machine learning approaches being implemented through the image processing unit 210, may allow an operator or related user to gain a better understanding of the quantity and nature of the defects. The recommended remedial actions associated with the cause may be provided to the user via the user interface 220 and/or the network connector 230. The welding system 100 may have the ability to take the recommended remedial actions for each type of classified defect. The welding system 100 may automatically implement the recommended remedial actions which may be immediately stopping the welding system 100 to prevent further production of a product which is defective or likely to be defective.

In further examples of defects, a row of continuous defects may be produced by a defect in the sonotrode 120 located at a certain position of the anvil roll 110. In this case, if the sonotrode 120 has an area that has failed, defective welds are generated all along the non-woven material 10. These can be easily detected by their continuous nature, allowing appropriate action to be readily taken. Significantly, the non-woven material inspection system 100 has the ability to take different actions for each type of classified defect. In this case, the system 300 may need to be immediately stopped as the product to which this material would later be adhered will likely fail. Also shown are random defects as previously described; these may result from the nature of the ultrasonic welding process, as well as from random variations in the material being used to form the discrete products. In addition, repeating defects as previously described are also shown, where similar thresholding may be applied in the image processing unit 210 in order to take the appropriate action, such as when these defects occur in significant enough quantity to warrant maintenance to the anvil roll 110 or the nest tool in the case of a discrete product manufacturing station. Significantly, knowing the location of pin damage in a position across the roll and at a certain rotational position relative to a zero (or reference) point allows only a particular point on the roll to be maintained and repaired. The means of locating the rotational position of the anvil roll 110 can be accomplished with an encoder, an additional camera imaging marks on the anvil roll 110 or other positional measurement methods such as magnetic encoders, rotary variable differential transformers (RVDTs) or Hall effect devices.

Another embodiment of the lighting 244 (e.g., independent lighting modules) actively lights both sides of the portion of the non-woven material 10 that is being imaged by the imaging unit 240. In this case, the lights are not built into the camera 242 but instead are mounted separately and can be adjusted to optimize the nature of the image for defect detection. These lights can be LED or fluorescent in order to create even illumination across the entire non-woven material 10. Appropriate lensing is added to spread the light out and ensure that there are no bright and dim spots; as such, wavelength, angle and placement may be varied in order to optimize defect recognition. Lighting can vary in width from 100 mm to 1000 mm wide depending on the application. As such, lighting 244 and the camera 242 may be mounted independently, such as to minimize interference with other parts of the machine vision system, such as (in certain configurations) the system 300. Likewise, multiple cameras 242 may be used.

In embodiments, the display (e.g., the user interface 220) may show defects on the non-woven material 10 highlighted by red outlines of dots where dots were expected and something else showed in the image. Defective images are shown in the bottom portion of the display, while live images of one revolution of the anvil roll 110 may be shown in the upper portion of the display. If the product being imaged is discrete, the entirety of the product would be shown in both the live and defect images. Although not shown, other display features (such as a pie chart or the like) may be used to show the distribution of defect types in order to guide the user to take appropriate action based on how many accumulated defects have occurred of each type, assuming the automated thresholds have not triggered specific alarms, marks or machine actions.

In embodiments, some bonds have grown larger and been combined with neighboring bonds indicating potential defects that have not yet reached threshold values. The same red dots for defects may be shown in the display as well. The diagnostic mode of the live image shown in the display may indicate how the image processing unit 210 defines and thresholds bonds to be able to detect if they are in their expected locations and well-formed. As before, red dots may be defects while green dots are correct (e.g., non-defective bonds). As shown, as the specific location has increasingly more defects, the red dots may become more intense on the display. As mentioned elsewhere, determination of what is a correct weld versus a defective one may be based on comparison with expected baseline images, whether though rule-based or machine learning-based embodiments of the image processing unit 210 and associated machine vision system components.

In embodiments, the display may show an image of the non-woven material 10. Based on the image, thickness or basis weight of the non-woven material 10 may be detected. As long as the dense areas and light areas of the image of the non-woven material 10 stay within a pre-determined range, the non-woven material 10 is considered to be substantially free of a defect. The image may be shown in “reverse” so that the darker areas represent thinner portions of the non-woven material 10, and the lighter areas represent thicker portions of the non-woven material 10. Thresholds of these light/dark values are determined and correlated with acceptable thickness of the material and appropriate defect classification takes place. The normal alarms, machine stops, marking and roll mapping can be made to take necessary action, based on the classified nature of these defects and recommended remedial actions.

In embodiments, the display may show defects on a roll map diagram. The defect viewer may show jobs collected. Each job may have a number of revolutions of the anvil roll 110 and those that have exceeded the threshold values for defects may be shown in a count with certain indicia (for example, a red X) and those that have not exceeded the threshold values may be shown with different indicia, such as a green check mark. Selecting one job for viewing, a film-strip view of each revolution of defects may be shown near the bottom of the display with a roll map below it to indicate exactly where in the roll (including certain measures of distance, such as feet or meters) the defect is located. The roll map can be uploaded to an unwind machine (not shown) that may use the product produced from the non-woven material 10 to stop the machine when the defect is reached; in one form, this may allow removal of this portion of the non-woven material 10.

In one aspect, defects may be identified by imaging the resulting product with a high resolution camera (e.g., the camera 242) and accompanying lighting (e.g., the lighting 244) driven by an encoder (e.g., the encoder 260) that in turn is driven by the rotation of the anvil roll 110. For example, one rotation of the cylinder of the anvil roll 110 is equal to one image of the product length when using the pixel-to-real-world value of length measurement. This produces an image that represents the product profile that encompasses the entire circumference of the anvil roll 110 where the product is in contact with the pins. Once the product has been imaged, the machine vision image processing chain will locate the resulting bonds, and produce a list of coordinate locations of these bond locations. This list is then compared to each subsequent image that also has the weld locations added to a list of coordinates. These two lists are compared to find differences of weld location coordinates, while taking into account any small variations in positional accuracy. Any bond location differences that cannot be resolved are classified as errors that in one form may be entered into yet another list of “previously reported errors”. This secondary list is used to plot trends in error location coordinates. When the next image is processed, if it contains errors, the error list of coordinates is compared to the “previously reported errors” list. If the listed coordinates match to a previously reported error location, a count is incremented for the reported coordinate location. This produces a count that represents repeating errors in the same coordinate location. If the “previously reported errors” list does not match to a new error coordinate location, the entry coordinate location count is reset on the list.

A roll is mapped utilizing this same methodology. A single rotation of the anvil roll 110 is exactly equal to the encoder 260 resolution programmed for a single revolution. Each encoder 260 signal generates a single line of a line-scan camera (e.g., the camera 244) image. In this way, each image length is equal to the entire circumference length of the anvil roll 110. The logging feature of the image inspection system 200 stores each image as an equivalent length of non-woven material 10 product. In this manner, the roll length is mapped with any quality issues noted for the length, that is in turn the equal length of the cylinder circumference of the anvil roll 110. A signal is sent from the image inspection system 200 at the completion of each roll. This signal is used by the image inspection system 200 to end the data logging for one roll, and to automatically begin logging for the next roll. This enables the log to also be read in reverse to allow for the roll to be placed upon a finishing system (not shown) for further alterations to the final product. This requires the roll to be identifiable, and linked to the associated log file. A label placed within the roll core, and or the outer layer of the completed roll achieves this link. This label contains the link to the associated log file located on a network.

On the defect image, the distribution of questionable sonic welds is shown with red dots and a histogram diagnostic tool aids in seeing how many defects are along one particular “column” of pixels. The filmstrip can be panned through toward the left going backward in the roll to see older defects in the job being investigated. The roll map can also be moved to select a portion of the location for closer examination. The images in the defect viewer can be more closely scrutinized by zooming in on the image.

In the embodiment with discrete products, the roll map would not be used, but the filmstrip view would page through images going backward in time and each image would represent a product manufactured with a defect detected by the vision system.

In embodiments, the image inspection system 200 may identify the outer edges of the non-woven material 10 and compare them to the learned total non-woven material 10 width. Further, the material laminated inside the sandwich of non-woven material may be identified. These edge measurements are subjected to the same tolerances that can be adjusted by the user to determine if the non-woven material 10 width and widths of other layers inside the laminate are correct.

In embodiments, the image inspection system 200 may assess material density identified from a different image of the surface of the non-woven material 10. Using the same principle as previously described, the image can be evaluated for material thickness and appropriate measures taken to reject thicknesses that exceeds set thresholds for thickness or thinness. In one form, the images are used to determine the quality of the product and if sufficient material is there for the welds to hold up when the non-woven material 10 is later used to assemble a product such as a sanitary napkin, diaper or absorbent pad as previously discussed.

In embodiments, a discrete product in the form of a face mask may be manufactured by the system 300, with ultrasonic welds around the mask perimeter. Green weld points may indicate as passable while yellow or red may indicate marginal but not yet defective on the image of the discrete product displayed. The discrete product evaluation operates identically to the aforementioned products except without the tracking of the roll map for defect storage. Defects may be stored as individual products, and in one form may be correlated to a particular pin (repeating), random (moving around on the product) or consistent across a large area (constant).

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

What is claimed is:

1. A method for inspecting a non-woven material comprising:
introducing, by a sonotrode, ultrasonic energy to weld the non-woven material;
supporting, by an anvil roll, the non-woven material between the anvil roll and the sonotrode;
rotating the anvil roll to transfer the non-woven material in a downstream direction;
taking, by an imaging unit disposed downstream of the anvil roll, an image of the non-woven material; and
processing the image by an image processing unit comprising:
identifying a defect based on the image;
classifying the defect into one of a plurality of categories;
analyzing a cause of the defect based on the respective category;
determining a component causing the defect; and
notifying the cause with a recommended remedial action associated with the component.

2. The method of claim 1, further comprising:
determining a position of the non-woven material by an encoder disposed upstream of the anvil roll.

3. The method of claim 1, wherein a length of the image corresponds to a length of one rotation of the anvil roll.

4. The method of claim 1, further comprising:
marking a location of the defect on the non-woven material by a marking valve disposed downstream of the imaging unit.

5. The method of claim 1, wherein the categories include a random defect that randomly appears on the non-woven material, a repeating defect that appears repeatedly in a pattern on the non-woven material, and a continuous defect that appears continuously on the non-woven material.

6. The method of claim 1, wherein the image processing unit is further configured to:
determine a degree of the defect; and
analyze the cause of the defect based on the degree of the defect.

7. The method of claim 6, wherein the degree of the defect is analyzed based on a degree of melting of the non-woven material.

8. The method of claim 1, wherein the image processing unit is further configured to:
determine a width of the non-woven material; and
analyze the cause of the defect based on the width of the non-woven material.

9. A method for inspecting defects on a non-woven material comprising:
introducing ultrasonic energy to weld the non-woven material;
transferring the non-woven material in a downstream direction;
taking an image of the non-woven material after welding the non-woven material;
identifying a defect based on the image;
classifying the defect into one of a plurality of categories;
analyzing a cause of the defect based on the respective category;
determining a component causing the defect; and
notifying the cause with a recommended remedial action associated with the component.

10. The method of claim 9, further comprising:
determining a position of the non-woven material before introducing the ultrasonic energy to the non-woven material.

11. The method of claim 9, further comprising:
marking a location of the defect on the non-woven material based on the identification of the defect.

12. The method of claim 9, wherein the categories include a random defect that randomly appears on the non-woven material, a repeating defect that appears repeatedly in a pattern on the non-woven material, and a continuous defect that appears continuously on the non-woven material.

13. The method of claim 9, further comprising:
determining a degree of the defect; and
analyzing the cause of the defect based on the degree of the defect.

14. The method of claim 13, wherein the degree of the defect is determined based on a degree of melting of the non-woven material.

15. The method of claim 9, further comprising:
determining a width of the non-woven material; and
analyzing the cause of the defect based on the width of the non-woven material.

16. A method for inspecting a non-woven material comprising:
introducing, by a sonotrode, ultrasonic energy to weld the non-woven material;
supporting, by an anvil roll, the non-woven material between the anvil roll and the sonotrode;
rotating the anvil roll to transfer the non-woven material in a downstream direction;
taking, by an imaging unit disposed downstream of the anvil roll, an image of the non-woven material;
processing the image by an image processing unit comprising:
identifying a defect based on the image;
classifying the defect into one of a plurality of categories;
analyzing a cause of the defect based on the respective category;
determining a component causing the defect; and
taking a recommended remedial action associated with the component.

17. The method of claim 16, wherein the categories include a random defect that randomly appears on the non-woven material, a repeating defect that appears repeatedly in a pattern on the non-woven material, and a continuous defect that appears continuously on the non-woven material.

18. The method of claim 16, wherein the image processing unit is further configured to:
determine a degree of the defect; and
analyze the cause of the defect based on the degree of the defect.

19. The method of claim 18, wherein the degree of the defect is analyzed based on a degree of melting of the non-woven material.

20. The method of claim 16, wherein the image processing unit is further configured to:
determine a width of the non-woven material; and
analyze the cause of the defect based on the width of the non-woven material.

21. The method of claim 1, further comprising:
detecting a basis weight or a dimension of the non-woven material; and
analyzing the cause of the defect based on the detected basis weight or dimension of the non-woven material.
